(19) United States
(12) Patent Application Publication
(10) Pub. No.: US 2026/0157772 A1
(43) Pub. Date: Jun. 11, 2026

(54) MEDICAL INTRODUCER SHEATH COMPRISING AN ULTRASONIC IMAGING PROBE

(71) Applicant: VERMON, TOURS (FR)

(72) Inventors: Tony MATEO, TOURS (FR); Guillaume FERIN, TOURS (FR); Maxime CHEPPE, TOURS (FR)

(21) Appl. No.: 19/407,797

(22) Filed: Dec. 3, 2025

(30) Foreign Application Priority Data
Dec. 5, 2024 (FR) 24/13507

(51) Int. Cl. A61B 17/34; A61B 8/00; A61B 8/12; A61B 17/00; B06B 1/06

(52) U.S. Cl. CPC A61B 17/3417; A61B 8/12; A61B 8/4488; A61B 8/4494; A61B 17/3415; B06B 1/0625; A61B 2017/00862; B06B 2201/76

(57) ABSTRACT

The present description relates to a medical introducer sheath extending in an axial direction between a distal end and a proximal end, and having an axial lumen and a bendable portion extending to the distal end, the bendable portion comprising an ultrasonic imaging probe comprising several ultrasonic transducers distributed in a matrix on a transverse face of the medical introducer sheath at the distal end around the axial lumen, an orientation coupler adapted to impart a curvature to the bendable portion, a sleeve disposed around the orientation coupler and comprising a tubular portion and a protruding peripheral portion wound in several protruding turns around the tubular portion along the axial direction, and connection strips extending from the transducers in the axial direction toward the proximal end and distributed around the sleeve, and an elastic structure wound in several turns around the sleeve and the connection strips.

### CROSS-REFERENCE TO RELATED APPLICATIONS

This application claims the benefit of and priority to French patent application number FR 24/13507, filed on Dec. 5, 2024, entitled “Gaine d'introduction médicale munie d'une sonde d'imagerie ultrasonore,” the entire contents of which is hereby incorporated herein by reference.

### TECHNICAL FIELD

The present description particularly relates to a medical introducer sheath, for example a trocar or a cannula, comprising an ultrasonic imaging probe integrated into a distal end of the medical introducer sheath.

The present description particularly relates to a medical introducer sheath, for example a trocar or a cannula, comprising an ultrasonic imaging probe integrated into a distal end of the medical introducer sheath.

The medical introducer sheath comprises, for example, an internal lumen adapted to receive a catheter or any other elongated medical device.

Certain techniques for exploration and intervention in anatomical regions, such as cardiac regions, use imaging, for example radioscopy or fluoroscopy (i.e. X-ray imaging), to help locate and guide medical devices to a target region of the patient, generally through the vasculature of the patient.

Medical devices that can be guided into a patient's body typically comprise access devices, or medical introducer sheaths, for example trocars or cannulas, diagnostic catheters and/or treatment catheters, for example ablation or replacement catheters.

Catheters and ultrasonic imaging probes have been developed to directly visualize the target region. For example, a catheter integrating an ultrasonic imaging probe, or imaging catheter, can be used to image the target region and then to guide an access device and a treatment catheter into that target region.

However, the imaging catheter is generally distinct from the access device and the treatment catheter. On the one hand, this requires multiple introduction/removal operations of the various devices, and even multiple access points, into the patient's body, with the risks and discomfort involved for the patient, and may lengthen de facto the intervention time. On the other hand, this requires to have a position tracking to track the location of each device in the patient's body, which is generally performed by fluoroscopy, which may increase the exposure of the patient and of the medical staff to X-rays. This may also require to make cross-checks of positioning between the different devices, which may be an additional source of time waste.

It would be desirable to have an exploration and intervention device that at least partially overcomes some of the disadvantages of known intracorporeal exploration and intervention devices.

In particular, there is a need for a medical introducer sheath that is adapted to receive a catheter or any other elongated medical device and that comprises an imaging functionality. It would be advantageous if the medical introducer sheath could be easily oriented so that it could be guided to a target anatomical region.

### SUMMARY OF THE INVENTION

One embodiment overcomes all or some of the disadvantages of known exploration and intervention devices.

One embodiment provides a medical introducer sheath extending in an axial direction between a distal end and a proximal end opposite the distal end, the medical introducer sheath having an internal axial lumen between said distal end and said proximal end, and a bendable portion extending to said distal end, the bendable portion comprising:
an ultrasonic imaging probe comprising several ultrasonic transducers distributed in a matrix on a transverse face of the medical introducer sheath at the distal end, around the axial lumen;
an orientation coupler adapted to impart a curvature to the bendable portion;
a sleeve disposed around the orientation coupler, said sleeve comprising a tubular portion and a protruding peripheral portion wound in several protruding turns around the tubular portion along the axial direction, the tubular portion being comprised between the peripheral portion and the orientation coupler; and
connection strips extending from the ultrasonic transducers in the axial direction toward the proximal end and distributed around the sleeve; and
an elastic structure wound in several turns around the sleeve and the connection strips, the elastic structure running between the protruding turns.

According to one embodiment, the peripheral portion has a helical shape, the protruding turns corresponding to the turns of the helix, the elastic structure also having a helical shape running between the protruding turns.

According to one embodiment, the protruding turns of the peripheral portion are protruding rings mutually disjoined and distributed, for example regularly distributed, over several circumferences of the tubular portion along the axial direction, the turns of the elastic structure also being rings mutually disjoined and positioned between the protruding rings.

According to one embodiment, the pitch between the turns of the elastic structure is substantially equal to the pitch between the protruding turns of the peripheral portion, the turns of the elastic structure being regularly distributed along the axial direction.

According to one embodiment, the pitch between the turns of the elastic structure is substantially equal to the pitch between the protruding turns of the peripheral portion, the turns of the elastic structure being irregularly distributed along the axial direction, for example with a higher density around a center of the bendable portion.

According to one embodiment, the axial lumen is sized to receive a catheter in a sliding and/or rotating manner.

According to one embodiment, the orientation coupler comprises links, for example ball joints, two adjacent links cooperating with each other so as to be able to pivot about at least one axis of rotation.

According to one embodiment, the orientation coupler comprises linkage cables connected to the links so as to hold the links against each other and to control the pivotal movement of the links, and thus a bending of the bendable portion.

According to one embodiment, the ultrasonic transducers of the ultrasonic probe are distributed in several concentric crowns around the axial lumen, each crown comprising several transducers distributed in radial sectors along said crown, for example the number of crowns is greater than 3 and the number of transducers per crown is greater than 30.

According to one embodiment, the ultrasonic transducers are piezoelectric transducers and are formed by several annular layers extending one over the other around the axial lumen, said annular layers comprising:
a piezoelectric layer metallized on each of its inner and outer faces and divided into several piezoelectric sectors, for example annular and radial sectors;
an impedance matching layer on the piezoelectric layer and divided into several impedance matching sectors, each impedance matching sector being positioned opposite a piezoelectric sector; and
an acoustic attenuation layer, beneath the piezoelectric layer.

According to one embodiment, the medical introducer sheath further comprises an interconnection structure including the connection strips, the interconnection structure further comprising an annular portion disposed between the acoustic attenuation layer and the piezoelectric layer, said annular portion comprising metallic tracks connected to the ultrasonic transducers and extending into the connection strips.

According to one embodiment, the medical introducer sheath further comprises an annular tip at the distal end, said tip being connected to the orientation coupler, for example to a distal link of said orientation coupler, the ultrasonic transducers being disposed in a circumferential groove of said tip all around the axial lumen.

According to one embodiment, the medical introducer sheath further comprises an outer envelope around the connection strips, the sleeve and the elastic structure, the outer envelope being, for example, made of a biocompatible material.

One embodiment provides an intracorporeal exploration and intervention device comprising a medical introducer sheath as described above.

According to one embodiment, the device further comprises a catheter configured to be introduced into the axial lumen of the medical introducer sheath.

According to one embodiment, the device further comprises, at the proximal end, a control handle adapted to control a bending of the bendable portion.

According to one embodiment, the medical introducer sheath is a trocar.

One embodiment provides a method of using the introducer sheath.

According to one embodiment, the using method comprises using the introducer sheath for the treatment of a cardiac condition, for example to implant a pacemaker, to perform a radiofrequency ablation or to replace or implant a heart valve.

### BRIEF DESCRIPTION OF THE DRAWINGS

The foregoing features and advantages, as well as others, will be described in detail in the following description of particular embodiments given by way of a non-limiting basis with reference to the accompanying drawings, among which:

FIG. 1 is an overview of an intracorporeal exploration and intervention device according to one embodiment;

FIG. 2A, FIG. 2B and FIG. 2C are longitudinal sectional views partially illustrating an example of the medical introducer sheath according to one embodiment;

FIG. 3A and FIG. 3B are three-dimensional views of the medical introducer sheath of FIGS. 2A to 2C;

FIG. 3C and FIG. 3D are views illustrating the interconnection structure of the medical introducer sheath of FIGS. 3A and 3B; and

FIG. 4 is a schematic view illustrating an example of an ultrasonic imaging probe of a medical introducer sheath according to one embodiment.

### DETAILED DESCRIPTION

Like elements have been designated by like references in the various figures. In particular, the structural and/or functional elements that are common among the various embodiments may have the same references and may dispose identical structural, dimensional and material properties.

For the sake of clarity, only the steps and elements useful for an understanding of the described embodiments have been illustrated and are described in detail. In particular, the ultrasonic transducers of the described ultrasonic probes have not been detailed, as the described embodiments are compatible with all or most known ultrasonic transducer structures.

Unless otherwise specified, when reference is made to two elements connected together, this signifies a direct connection without any intermediate elements other than conductors, and when reference is made to two elements coupled together, this signifies that these two elements can be connected or coupled via one or more other elements.

Unless otherwise specified, when reference is made to two elements mounted, or positioned, on top of each other, this does not necessarily signify that these two elements are mounted, or positioned, directly on top of each other, as one or more other elements may be positioned between these two elements.

In the following description, unless otherwise specified, when reference is made to absolute positional qualifiers, such as the terms “front”, “back”, “top”, “bottom”, “left”, “right”, etc., or to relative positional qualifiers, such as the terms “above”, “below”, “higher”, “lower”, etc., or to qualifiers of orientation, such as the terms “horizontal”, “vertical”, etc., reference is made to the orientation in the figures.

Unless otherwise specified, the expressions “around”, “approximately”, “substantially” and “in the order of” signify within 10% or 10°, and preferably within 5% or 5°.

In the following description, unless otherwise specified, when reference is made to a transducer, reference is made to an ultrasonic transducer, when reference is made to a probe, reference is made to an ultrasonic imaging probe and when reference is made to a sheath or to an introducer sheath, reference is made to a medical introducer sheath. A medical introducer sheath may, for example, be a trocar or a cannula, or any other medical instrument for forming a pathway into an anatomical region.

In the following description, when reference is made to a catheter, reference is made, in a broad sense, to a thin, hollow or solid rod-shaped device, generally comprising at least one bendable portion, and to be introduced into a region of a human or animal body (for example a cavity, a lumen or a duct).

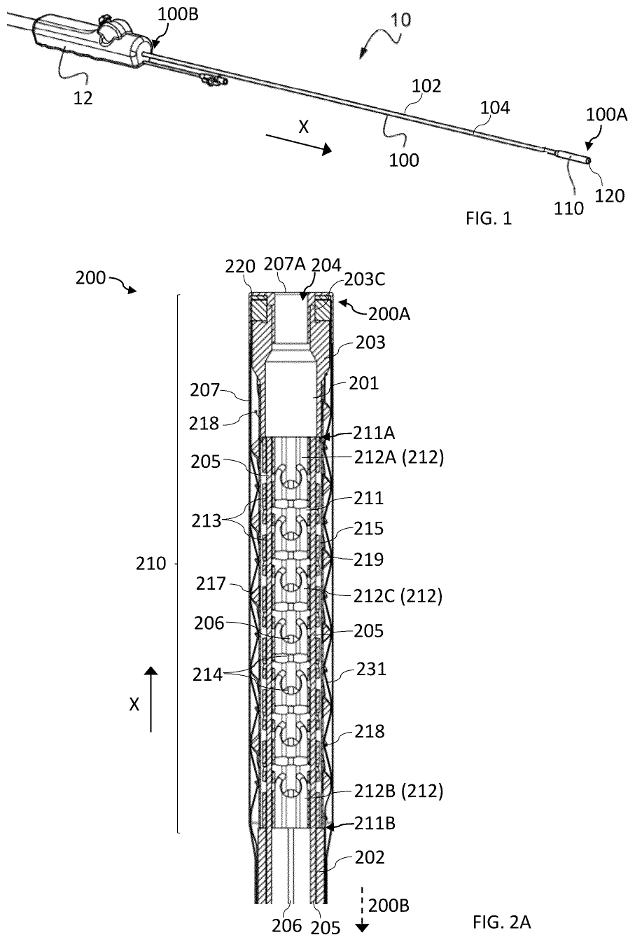

FIG. 1 is an overview of an intracorporeal exploration and intervention device 10 according to one embodiment.

The device 10 comprises a medical introducer sheath 100 and a control handle 12 connected to the proximal end 100B of the sheath 100. The sheath 100 is to be introduced into a human or animal anatomical region, for example to explore and/or intervene in that anatomical region.

The sheath 100 has, along its length, a shape of a tubular rod 102, defining an axial lumen 104 inside the tubular rod 102. The axial lumen 104 is adapted to receive a catheter or any other elongated medical device (not illustrated). For example, the internal axial lumen 104 is sized to receive a catheter in a sliding and/or rotating manner. The length of the catheter may be substantially equal to the length of the sheath 100 and of the tubular rod 102.

The sheath 100 comprises a bendable portion 110, or orientable portion, i.e. a portion that can be curved to adapt to the anatomy of the region into which it is introduced, for example, up to an angle between 90° and 180° relative to the axial direction X. The bendable portion 110 extends over a portion of the sheath 100 up to the distal end 100A of the sheath 100. The bendable portion 110 has, for example, a length of a few centimeters (cm), for example of approximately 4 cm.

The control handle 12 is adapted to control the advancement and the positioning of the sheath 100, as well as the bending, or curvature, of the bendable portion 110 of the sheath 100.

The sheath 100 comprises, at its distal end 100A, which is also the distal end of the bendable portion 110, an ultrasonic imaging probe 120. For example, the probe 120 extends all around the axial lumen 104 at the distal end 100A.

Throughout the description, the term “proximal” (or “rear”) is considered in relation to the device as a whole, i.e. in the direction of the control handle, and the term “distal” (or “front”) refers to an opposite direction, toward the exploration and/or intervention region (target region). The distal end of the medical introducer sheath corresponds to the end via which this sheath is introduced into the target region and the proximal end corresponds to the end opposite the distal end.

Throughout the description, the term “axial” is used in reference to the axis, in the X direction, of the device 10, i.e. its largest dimension (length), also corresponding to the largest dimension (length) of the sheath 100, and a “radial” direction is a direction lying in a plane perpendicular to the axial direction X. The term “longitudinal” refers to a direction parallel to the axial direction X, and the term “transversal” refers to a plane or a direction perpendicular to the axial direction X. A longitudinal section is a section made in a plane including the X direction, while a transverse section is a section made in a plane perpendicular to the X direction including the radial direction.

The probe 120 is oriented in a forward-looking manner, i.e. oriented from the distal end 100A of the sheath 100 in the direction of advancement of the sheath 100. In other words, the sheath 100 comprising the probe 120 at its distal end 100A is capable of transmitting and receiving ultrasonic signals in a direction generally oriented towards the front. The probe 120 allows to visualize the anatomical regions located opposite the distal end 100A of the sheath 100. Especially, the probe 120 allows to visualize the target anatomical region during the intervention, or even before and/or after the intervention, without having to introduce another catheter comprising an imaging probe.

For example, the probe 120 can provide real-time ultrasonic images of anatomical regions in the direction of advancement of the sheath 100 through the patient's body. For example, the ultrasonic images generated from the probe 120 can be used to guide the sheath 100 to the target region, to confirm the tissue contact of a catheter in the target region, to determine the orientation of the sheath 100 and/or the catheter in the patient's body, to monitor the progression of a lesion being formed in the tissue, or even to monitor the adjacent anatomical structures, for example to avoid undesirable side effects on these structures, to monitor the progression and the effectiveness of the treatment, etc.

Advantageously, the sheath 100 comprising the probe 120 can be used to deploy a catheter in the target anatomical region. Advantageously, the sheath 100 comprising the probe 120 can significantly reduce, or even eliminate, the use of radioscopy as a means of visualizing the sheath 100 and the catheter during an intervention. In other words, the ultrasonic guidance provided by the sheath 100 makes it possible to limit, or even to get rid of, the radioscopic guidance for the introduction of the sheath to a target region.

In a particular, non-limiting, form of implementation of the embodiments, the sheath 100 can be used, in combination with a catheter positioned in the axial lumen 104, to implant a pacemaker, to perform a tissue ablation by radiofrequency (RF ablation) or by cryoablation, or to replace or implant a heart valve, for example to perform a transcatheter aortic valve implantation (TAVI) or a transcatheter aortic valve replacement (TAVR). However, the embodiments are not limited to these uses or to any specific clinical use.

The probe 120 comprises, for example, an array of ultrasonic transducers, preferably in the form of a matrix of ultrasonic transducers.

An ultrasonic transducer is a transducer adapted to convert an electrical signal into an ultrasonic wave and conversely, to convert an ultrasonic wave into an electrical signal. Depending on the type of transducer, the electrical signal may correspond to a voltage, a current or an electric charge.

The transducer array may comprise any type of ultrasonic transducers, or even several types of ultrasonic transducers.

Ultrasonic transducers may consist of a layer of single-crystal or polycrystalline piezoelectric material, for example PZT (lead-zirconia titanate), or of a composite structure comprising at least one layer of piezoelectric material, for example a layer of PZT including polymer-filled grooves.

Ultrasonic transducers can be Micro-Electro-Mechanical Systems, or MEMS, which implement microelectronics production technologies. A MEMS-type transducer generally comprises one or more acoustic elements, each comprising one (or more) deformable membrane(s) suspended above a cavity and connected by a common electrode. According to one embodiment, each deformable membrane is moved or deformed by a capacitive effect with an electrode attached to that membrane and an electrode separated by the cavity. This type of ultrasonic transducer is known by the acronym CMUT, which stands for Capacitive Micro-machined Ultrasonic Transducer, or a membrane capacitive transducer. According to another embodiment, each deformable membrane is moved or deformed by a piezoelectric effect with a layer of piezoelectric material comprising two electrodes attached to the membrane. This type of ultrasonic transducer is known by the acronym PMUT, which stands for Piezoelectric Micro-machined Ultrasonic Transducer, or membrane piezoelectric transducer.

There is the issue of establishing a connection, particularly an electrical, or even optical, connection, between the ultrasonic transducers of the ultrasonic imaging probe 120 at the distal end 100A of the sheath and the proximal end 100B of the sheath 100 where the control handle 12 is located.

The ultrasonic transducers are generally connected to an interconnection structure at the probe 120 and in particular, to conductive tracks, generally metallic, of the interconnection structure, the interconnection structure extending via connection (conductive) strips, for example cables, layers, blades or ribbon cables, of connection, connected to the conductive tracks of the interconnection structure. The connection strips extend in the axial direction X toward the proximal end 100B of the sheath 100.

A major difficulty in achieving the connection is related to the bending of the sheath 100, at least of the bendable portion 110 of the sheath 100 which undergoes significant and opposing deformations between the inside and the outside of the bending. Ideally, the connection strips should pass through the center of the sheath 100, i.e. in the internal axial lumen 104 of the sheath 100, preferably as close as possible to the axis of the sheath 100, so as to minimize the stresses/deformations associated with the distance from the neutral line when the sheath 100 is bent. However, since the axial lumen 104 is for the passage of a catheter or of any other elongated medical device, the connection strips cannot pass through the axial lumen 104 and they pass around the periphery of the sheath 100. Thus, the connection strips move away from the axis of the sheath 100 and from the neutral line, especially all the more so that the diameter of the sheath 100 is large.

When the sheath 100 is bent, one hemicylindrical half is subjected to an elongation (extension) on the side opposite the curvature while the other half is subjected to contraction (compression) with a same amplitude. The elongation and the contraction generated by the curvature are greater the further away from the axis of the sheath 100 and the closer to the plane of curvature. The connection strips passing around the periphery of the sheath 100, on the plane of curvature, also undergo the elongation and the contraction generated by the curvature. One existing solution to prevent the connection strips from undergoing these stresses, or at least to limit them, is to wrap them around the sheath 100, for example in a substantially helical manner, in order to homogeneously distribute the areas subject to the elongation and the contraction. However, in addition to the potential technical difficulty of implementing this type of solution within the constraining dimensions of the introducer sheaths, ranging from a few millimeters to a few tens of millimeters in outer diameter, winding inevitably leads to the elongation of the connection strips, which in turn leads to increase electrical losses in proportion.

Furthermore, in the case of transducer arrays with a high-density of transducers, the interconnection of the transducers is generally at high-density, within small and constrained dimensions, which can amplify electrical losses and generally increase costs.

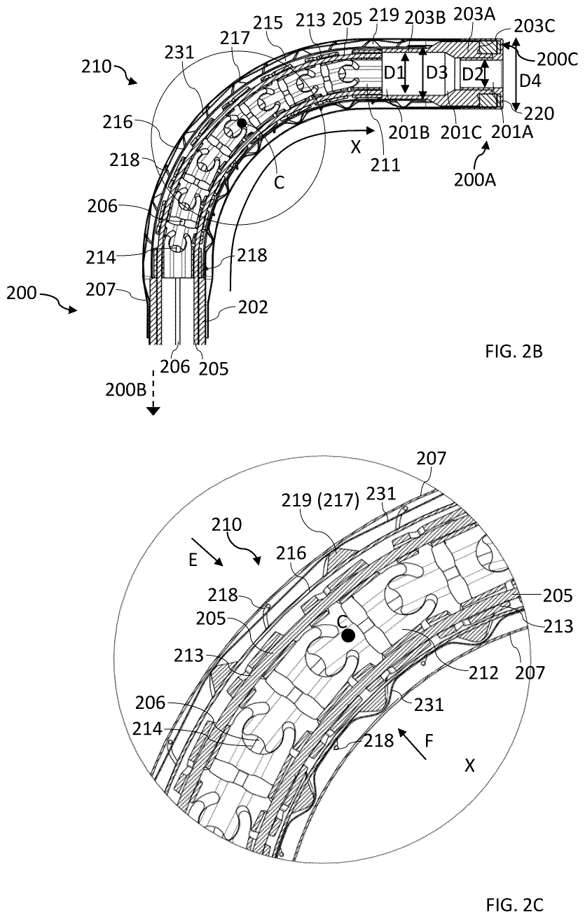

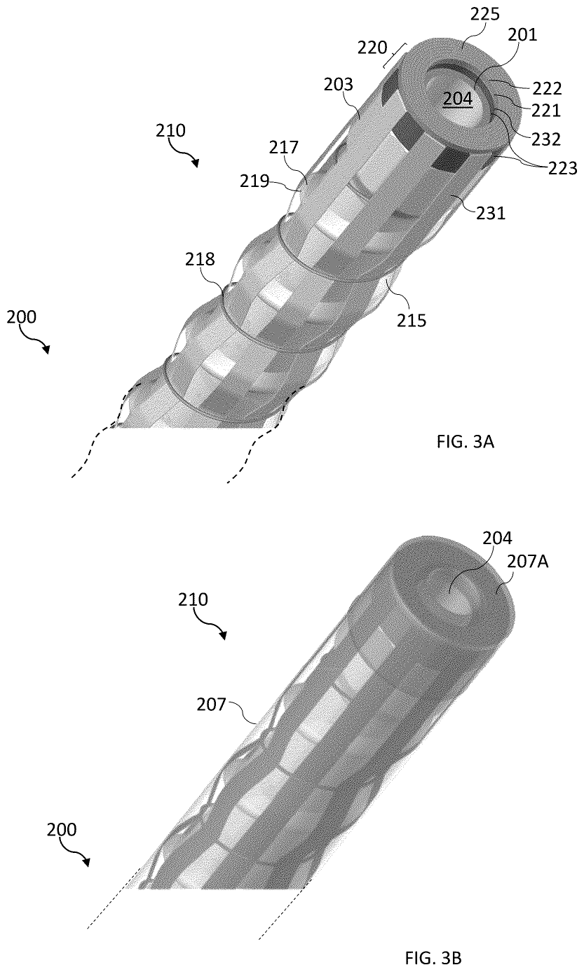

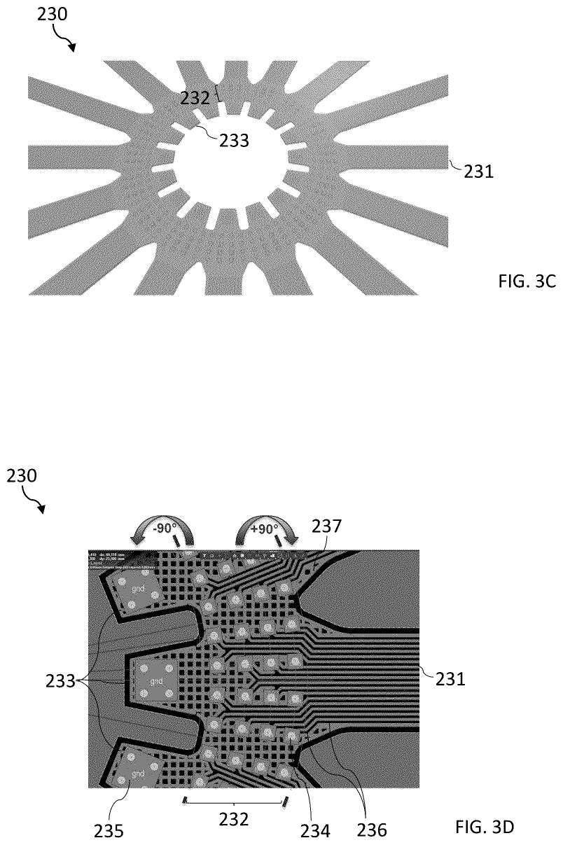

FIGS. 2A to 3D below illustrate a solution addressing the issue of establishing a connection between the ultrasonic transducers of the ultrasonic imaging probe 120 and the control handle 12, which allows the problems of elongation and contraction generated by the curvature of the sheath 100 to be managed while limiting the length of the connection strips, thereby reducing the associated electrical losses.

In addition, a solution is sought to address the issue of elongation and contraction generated by the curvature of the sheath, even with a transducer array with a high-density of transducers and a high-density of interconnections allowing an individual or RCA-type addressing of these transducers.

FIG. 2A, FIG. 2B and FIG. 2C are longitudinal sectional views partially illustrating an example of a medical introducer sheath 200 according to one embodiment. FIGS. 2A to 2C illustrate more specifically the bendable portion 210 of the introducer sheath 200, or distal portion 210. FIG. 2A is a view of the distal portion 210 in a straight configuration. FIG. 2B is a view of the distal portion 210 in a configuration curved at about 90°. FIG. 2C is a detailed view of the distal portion 210 taken from the circle illustrated in FIG. 2B.

When the sheath is curved, it can be referred to as a crutched sheath in the technical field of the present description.

In FIG. 2A, the axial direction X is straight while in FIGS. 2B and 2C, the axial direction X is bent.

The introducer sheath 200 illustrated in FIGS. 2A to 2C may correspond to the sheath 100 in FIG. 1, with the distal portion 210 then corresponding to the bendable portion 110 in FIG. 1.

The sheath 200 is hollow, with an internal axial lumen 204 extending along the axial direction X between the distal end 200A and the proximal end 200B of the sheath 200.

The distal portion 210 of the medical introducer sheath 200 comprises an orientation coupler 211, which is adapted to impart a curvature to the distal portion 210.

The orientation coupler 211 comprises a plurality of links 212, two adjacent links cooperating with each other so as to be able to pivot about at least one axis of rotation. For example, the links 212 form a joint.

The plurality of links 212 comprises in particular:
a distal link 212A at the distal end 211A of the coupler 211;
a proximal link 212B at the proximal end 211B of the coupler 211; and
intermediate links 212C between the distal link 212A and the proximal link 212B.

The links 212 are, for example, made of a material likely to constitute plain bearings between the links, for example:
a metal: for example a steel, aluminum, tungsten, titanium, etc.;
a rigid polymer material: for example a polycarbonate (PC), a polymethyl methacrylate (PMMA), etc.;
a ceramic material: for example alumina (Al₂O₃), a zirconium oxide (ZrO₂), a silicon carbide (SiC), etc.

The links 212 are, for example, ball joints. A person skilled in the art will be able to determine other types of links capable of pivoting relative to each other around at least one axis of rotation, so as to impart a curvature to the bendable portion 210 of the sheath 200.

The distal link 212A is connected to a distal ring 201. The proximal link 212B is connected to a proximal ring 202, for example is fitted into the proximal ring 202. The rings 201 and 202 may form stiffeners. The distal ring 201 may be referred to as the “head of crutching”.

The distal ring 201 is itself fitted into a tip 203 that is engaged with the distal link 212A, so as to form an integral distal ring/tip/distal link assembly. The tip 203 is for example an annular tip.

In the example illustrated, the distal ring 201 has a diameter that tapers toward the distal end 200A of the sheath 200. Thus, the distal ring 201 comprises a cylindrical proximal portion 201B with a diameter D1, a cylindrical distal portion 201A with a diameter D2 smaller than the diameter D1 and a frustoconical portion 201C connecting the portions 201A and 201B.

In the example illustrated, the tip 203 has a diameter that increases toward the distal end 200A of the sheath 200. Thus, the tip 203 comprises a cylindrical proximal portion 203B with a diameter D3 (which is engaged with the distal link 212A), a cylindrical distal portion 203A with a diameter D4 greater than the diameter D3 and a frustoconical portion connecting the portions 203A and 203B.

The rings 201, 202 and the tip 203 are hollow, for example annular, and the links 212 are ring-shaped, or at least are shaped so as to delimit a hollow central portion, such that the sheath 200 retains an axial lumen 204, including in the distal portion 210.

The links 212 are held against each other by cables 205, respectively 206, or by linkage cables, for example metallic cables, which pass through grooves 213, respectively 214, formed on peripheries of the links 212.

Two cables 205 constitute a first pair of cables parallel to each other in the plane of FIGS. 2A to 2C, while two other cables 206 constitute a second pair of cables parallel to each other in a plane perpendicular to the plane of FIGS. 2A to 2C. For example, the cables 205 allow the distal portion 210 to be curved in a direction perpendicular to the X direction and a plane parallel to the plane of FIGS. 2A to 2C, and the cables 206 allow the distal portion 210 to be curved in a direction perpendicular to the X direction and perpendicular to the plane of FIGS. 2A to 2C.

For example, the cables 205 and 206 each have a distal end retained in the distal link 212A, in the ring 201 or in the tip 203.

The cables 205 and 206 preferably extend to the proximal end 200B of the sheath 200, for example to the control handle 12 visible in FIG. 1. The cables 205 and 206 can thus be controlled in order to control the pivotal movement of the links 212, and thus the bending of the distal portion 210.

The distal portion 210 of the medical introducer sheath 200 further comprises a sleeve 215, which is disposed around the orientation coupler 211, i.e. around the links 212.

The sleeve 215 can be engaged, while being around the orientation coupler 211, with the proximal ring 202. The sleeve 215 can extend around the tip 203, for example around the proximal portion 203B of the tip 203.

The sleeve 215 is made of a material that allows flexibility, so that the sleeve 215 can follow the curvature of the distal portion 210. For example, the sleeve 215 may be made of an elastomeric material, for example a thermoplastic elastomer (TPE), for example of a polyether block amide (PEBA), for example of silicone. The sleeve 215 may be made of a biocompatible material, but this is not mandatory, as the sleeve is not in contact with the external environment of the sheath 200, i.e. with the environment surrounding the sheath 200 or inside the sheath 200, in the lumen 204.

The sleeve 215 comprises a substantially cylindrical tubular portion 216 and a peripheral portion 217 protruding from the outer wall of the tubular portion 216. The peripheral portion 217 comprises a plurality of protruding turns 219, or protrusions. The protrusions 219 protrude in the radial direction outward, i.e. away from the axis, so that the tubular portion 216 is comprised between the protrusions 219 and the links 212.

In the example illustrated, the peripheral portion 217 has a helical, protruding, shape that winds around and along the tubular portion 216 in the axial direction X. The protrusions 219 correspond to the turns of the helix and are thus connected to each other. The pitch of the helix may be regular or irregular.

As a variant, the peripheral portion may comprise several protruding rings that are disjoint from each other and wrapped around the tubular portion 216. The protruding rings are distributed, for example regularly distributed, over several circumferences of the tubular portion 216 along the axial direction X, with the protruding rings forming the protrusions. In this case, the protruding turns forming the protrusions are disjoint from each other.

The sheath 200 comprises, at its distal end 200A, which is also the distal end of the distal portion 210, an ultrasonic imaging probe 220 called forward-looking ultrasonic imaging probe, i.e. oriented from the distal end 200A of the sheath 200 in the direction of advancement of the sheath 200, as defined above. The probe 220 extends all around the axial lumen 204 on the transverse face 200C of the sheath 200 at the distal end 200A. The transverse face 200C is a face perpendicular to the axial direction X. The transverse face 200C is in this example in the form of a crown, and is all around the axial lumen 204.

The probe 220 comprises an array of several ultrasonic transducers 225 (marked in FIG. 3A) which are positioned at least partly in a circumferential groove 203C formed in the tip 203 all around the axial lumen 204.

The array of transducers is preferably a transducer matrix.

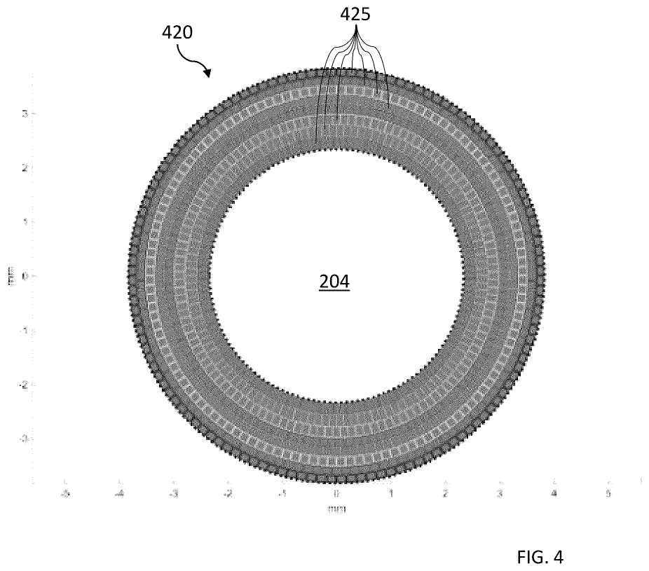

The transducer array may comprise any number of ultrasonic transducers, for example between 128 and 1024, it may for example comprise several concentric crowns each comprising several ultrasonic transducers. The ultrasonic transducers of a same crown may be distributed in radial sectors of the crown, as illustrated in the example in FIG. 4 described below.

For example, the number of crowns is greater than 3 and the number of transducers (radial sectors) per crown is greater than 30.

The transducers are generally connected to an interconnection structure 230 (described below in connection with FIGS. 3A to 3D) at the probe 220 and in particular, they are connected to conductive tracks of the interconnection structure. The interconnection structure is extended by connection strips (conductive) 231, for example connection cables, layers, blades or ribbon cables connected to the conductive tracks of the interconnection structure. In the rest of the description, the connection strips 231 are referred to as ribbon cables 231.

The ribbon cables 231 are each made, for example, from a flexible printed circuit. A flexible printed circuit consists of conductive tracks, for example made of copper, arranged on or inside a flexible insulating substrate made of a dielectric material, generally a polymer, for example polyimide.

The ribbon cables 231 extend in the axial direction X from the probe 220 toward the proximal end 200B of the sheath 200. For example, the ribbon cables 231 are substantially straight in the configuration illustrated in FIG. 2A (non-curved sheath).

The ribbon cables 231 pass around the periphery of the sheath 200, in particular around and along the sleeve 215. For example, the ribbon cables 231 are regularly distributed around the sleeve 215.

The ribbon cable 231 may extend to the proximal end 200B of the sheath 200. As a variant, the ribbon cables 231 may terminate before the proximal end 200B of the sheath 200 and, for example, be connected to a flexible conductive layer that extends to the proximal end 200B, or even beyond.

The distal portion 210 of the medical introducer sheath 200 further comprises an elastic structure 218 wound around the sleeve 215 and the ribbon cables 231. The elastic structure 218 runs along several circumferences between the protrusions 219.

The elastic structure 218 is adapted, when the distal portion 210 is curved, to absorb the elongation (extension) of one side E (side opposite the curvature, or side of the large radius of curvature) of the sheath 200, and the contraction (compression) of the other side F (side of the curvature, or side of the small radius of curvature) of the sheath 200, with the ribbon cables 231 remaining extended in the axial direction X, i.e. without having to wrap the ribbon cables 231 around the sheath 200, thus making it possible to limit the lengths of the ribbon cables and thereby to limit electrical losses in proportion.

As can be seen in FIG. 2C:
on the side E opposite the curvature, the ribbon cables 231 are substantially stretched between the protrusions 219 and the elastic structure 218 is extended on this side E; and
on the side F of the curvature, the elastic structure 218 in compression presses the ribbon cables 231 against the sleeve 215, and in particular against the protrusions 219 and against the tubular portion 216 between the protrusions 219.

Thus, the protrusions 219, by the profile they form with the tubular portion 216, combined with the elastic structure 218, allow the ribbon cables 231 to all have substantially a same length. A person skilled in the art will be able to determine the thickness, and possibly the width in the X direction, of the protrusions 219 in order to achieve this effect.

The elastic structure 218 is made of a material that is sufficiently soft to be wrapped around the ribbon cables 231, for example of rubber, of an elastomer or of a polymer.

The cross-section of the elastic structure 218 is, for example, circular, oval, rectangular or polygonal. The elastic structure 218 may also consist of a rigid wire structured in the form of a spring.

It should be noted that the elastic structure 218 remains with a relatively low degree of deformation when moving from a straight configuration (FIG. 2A) to a curved configuration (FIG. 2B). In fact, the elastic structure 218 moves away from the center of curvature without being significantly deformed. The elasticity of the structure 218, as well as its cross-section, should preferably be adapted by a person skilled in the art to absorb the deformations of the ribbon cables 231.

In the example illustrated, the elastic structure 218, as well as the peripheral portion 217, is in the form of a helix with several turns, running around and along the sleeve 215 in the axial direction X. The turns of the elastic structure 218 run between the protrusions 219 of the peripheral portion 217.

As a variant, the elastic structure, and in this case the peripheral portion, may be in the form of several rings that are disjoint from each other. In this case, the turns of the elastic structure are disjoint from each other. The rings of the elastic structure are positioned between the protruding rings of the peripheral portion.

The turns of the elastic structure 218 and the turns of the peripheral portion 217, whether they are joined or disjoined in the form of rings, may be distributed at regular intervals in the X direction.

As a variant, the turns of the elastic structure 218 and the turns of the peripheral portion 217, whether they are joined or disjoined in the form of rings, may be irregularly distributed, for example with an increased density around the center C of the distal portion 210, where the curvature reaches its maximum.

The number of turns of the elastic structure 218 is, for example, between 5 and 150. More generally, a person skilled in the art will be able to determine the number of turns depending on the angle of flexion of the distal portion 210, the outer diameter of the sheath 200, the radius of curvature and the absorption capacity of the protrusions 219. The skilled person may choose to distribute the elastic structure 218 over substantially the entire length of the sheath 200 and not only around the bendable portion 210, which may increase the number of turns.

An outer envelope 207 in the form of a soft sheath envelops all of the elements described above, in particular the ribbon cables 231, the elastic structure 218, the sleeve 215, the rings 201, 202, the tip 203 and the cables 205, 206.

This outer envelope 207 may comprise a distal end 207A positioned on the probe 220, and in this case, the outer envelope 207 is preferably transparent to ultrasonic waves, for example is made of silicone.

The outer envelope 207 is preferably biocompatible, for example is made of silicone, for example of PEBA, or is coated with a biocompatible material, for example is coated with parylene.

An inner envelope (not visible in FIGS. 2A to 2C) may be provided around the distal portion 201A of the distal ring 201, the distal portion 203A of the tip 203 then being around this inner envelope. The inner envelope is, for example, made of silicone.

As a non-limiting illustration, the largest diameter of the axial lumen 204, i.e. the internal diameter of the distal portion 210 of the sheath 200, is between 2.5 and 25 mm, and the overall diameter of the distal portion 210 of the sheath 200, i.e. the external diameter of the outer envelope 207, is between 7 and 30 mm.

FIG. 3A and FIG. 3B are three-dimensional views of the medical introducer sheath 200 of FIGS. 2A to 2C. FIG. 3C and FIG. 3D are views illustrating the interconnection structure 230 of the medical introducer sheath of FIGS. 3A and 3B. FIG. 3A illustrates in a three-dimensional view the bendable portion 210 without the outer envelope, while FIG. 3B illustrates in a three-dimensional view the bendable portion 210 with the outer envelope 207. FIG. 3C illustrates in a three-dimensional view a detail of the interconnection structure 230 deployed perpendicular to the X direction. FIG. 3D illustrates another detail of the interconnection structure 230 in a direction parallel to the X direction.

The introducer sheath illustrated in FIGS. 3A and 3B corresponds to the sheath 200 in FIGS. 2A to 2C, with the bendable portion corresponding to the distal portion 210 in FIGS. 2A to 2C.

In the example of FIG. 3A, the ultrasonic transducers 225 of the ultrasonic imaging probe 220 are transducers based on piezoelectric material and are formed by several annular layers extending one over the other around the axial lumen 204:
a layer of piezoelectric material 221, or piezoelectric layer 221, cut through its entire thickness to form several sectors of the piezoelectric layer, or piezoelectric sectors: the piezoelectric layer may be metallized on each of its inner and outer faces to form an outer metallic layer and an inner metallic layer, cutting of the piezoelectric layer including cutting of the external and internal metallic layers, each piezoelectric sector thus comprising an external electrode, corresponding to a sector of the cut external metallic layer, and an internal electrode, corresponding to a sector of the cut internal metallic layer; and
an impedance matching layer 222 on the piezoelectric layer: the impedance matching layer is cut, generally at the same time as the piezoelectric layer, to form several sectors of the impedance matching layer, or impedance matching sectors, each impedance matching sector being positioned opposite a piezoelectric sector, and for example in contact with that piezoelectric sector, forming a stack.

Instead of a single piezoelectric layer, there may be a stack of piezoelectric layers.

A stack of an impedance matching sector on a piezoelectric sector makes it possible to form all or part of an ultrasonic transducer 225. Stacks of impedance matching and piezoelectric sectors are generally separated from each other by slots, or kerfs.

The sectors may be annular and radial, such that the transducer array comprises crowns, each comprising several transducers 225 distributed along the circumference of the crown.

An annular layer of an acoustic damping material 223, or backing layer, is positioned beneath the piezoelectric layer 221. The backing layer 223 is, for example, not cut into sectors.

The interconnection structure 230 comprises an annular portion 232 sandwiched between the backing layer 223 and the piezoelectric layer 221 and connected to the ribbon cables 231.

The ribbon cables 231 are folded over the tip 203 and the sleeve 215.

As illustrated in FIGS. 3C and 3D, the annular portion 232 comprises electrical contact pads 234 each of which being connected to a metallic track 236. Generally, a contact pad 234 is connected to a transducer 225. The metallic tracks 236 extend into the ribbon cables 231. The contact pads 234 and the metallic tracks 236 are, for example, dedicated to the transducer signals. The metallic tracks 236 are insulated from each other and arranged in, and/or on, an insulating support 237, or dielectric support. The dielectric support 237 is, for example, in the form of a film of polymer material, preferably flexible, for example of polyimide. Several other materials are suitable for a flexible dielectric support, for example a polyester, a poly(ethylene naphthalate) or a polyetherimide. The metallic tracks 236 may advantageously be made of a malleable material, for example of gold or copper. This is because the metallic tracks 236 are folded at the same time as the ribbon cables 231. In addition, the interconnection structure 230 comprises internal strips 233, or tabs 233. Each tab 233 comprises an electrical contact pad 235, which is, for example, a ground pad.

The tabs 233 are folded inside the sheath 200, i.e. in the axial lumen 204, for example in contact with the inner wall of the distal ring 201.

The annular portion 232 is connected to the ribbon cables 231 and the tabs 233, and is located between the ribbon cables 231 and the tabs 233 which extend radially in two opposite directions from each other from the annular portion 232.

For example, the number of tabs 233 is equal to the number of ribbon cables 231.

According to an example of implementation, the interconnection structure 230 comprises sixteen ribbon cables 231 and eighteen metallic tracks 236 per ribbon cable (two for the ground and sixteen connected to the electrodes of the transducer elements of the ultrasonic probe 220), allowing 256 transducers to be connected electrically independently.

The metallic tracks 236, for example, have a width of about 20 μm and are spaced apart by about 50 μm, with a ribbon cable width of approximately 2 mm. It is possible to produce metallic tracks that are thinner and tighter between them. For example, the metallic tracks 236 may have a width of less than 15 μm, for example equal to about 5 μm, and a spacing of less than 25 μm, for example equal to about 5 μm. This may allow more transducers to be electrically connected, for example more than the 256 transducers indicated in the example of the interconnection structure 230.

According to another solution for electrically connecting more transducers, which may be combined with the previous solution, the interconnection structure 230 may comprise several interconnection layers, on and/or in the dielectric support 237 and each interconnection layer may be similar to that described above, and the metallic tracks of the different interconnection layers being connected to each other by vertical connections called “vias.” This makes it possible to interconnect a very large number of transducers, typically more than 256, for example 512 transducers for two interconnection layers similar to that described above, 768 transducers for three interconnection layers similar to that described above, 1024 transducers for four interconnection layers similar to that described above . . .

The interconnection structure 230 may be a flexible printed circuit board, or “FPCB.”

FIG. 3B illustrates that the outer envelope 207 may be extended over the probe 220 (portion 207A), as described further below, and that it may also be extended inside the axial lumen 204, in contact with the inner wall of the sheath 200.

FIG. 4 is a schematic view illustrating an example of an ultrasonic imaging probe 420 of a medical introducer sheath according to one embodiment.

The ultrasonic imaging probe 420 may correspond to the ultrasonic imaging probe 220 of FIGS. 2A, 2B and 3A. The medical introducer sheath may correspond to the sheath 200 of FIGS. 2A to 2C.

In the ultrasonic imaging probe 420, the transducer array comprises several concentric crowns around the axial lumen 204, each crown comprising several transducers 425. In this example, the transducer array comprises a same number of transducers for all crowns, a substantially equal surface area for all transducers, while having a substantially constant spacing between the transducers. In the example illustrated, the transducer array comprises eight crowns, and 128 transducers per crown, forming a matrix of 1024 transducers.

In the ultrasonic imaging probe 420, a first electrode of each transducer 425 is individually connected to a conductive track of the interconnection structure 230 and a second electrode is connected to a common ground with the other second electrodes of the other transducers of the probe 420, so that each transducer can be driven individually.

As a variant, the first electrodes of the transducers 425 of a same angular sector may be connected to each other, and the second electrodes of the transducers of a same crown may also be connected to each other. This results in a transducer matrix drivable by radius-angle whose driving principle is similar to the matrices addressable by row-column (RCA). This variant has the advantage of reducing the number of connections required for an equivalent number of transducers, or of increasing the number of transducers for an equivalent number of connections. In the example in FIG. 4, the probe 420, for example, would then require 8 plus 128, or 136 connections instead of 1024.

The described examples of implementation show that it is possible to have a medical introducer sheath adapted to receive a catheter or any other elongated medical device and which comprises an imaging functionality. In addition, the medical introducer sheath can be oriented while allowing electrical connection of the ultrasonic transducers.

The medical introducer sheath according to the embodiments can find applications in the field of the treatment of heart conditions, for example to implant a pacemaker, to perform a radiofrequency ablation (RF ablation) or to replace or implant a heart valve, for example to perform a transcatheter aortic valve implantation (TAVI) or a transcatheter aortic valve replacement (TAVR), the sheath being generally used in combination with a catheter or any other elongated medical device positioned in the axial lumen of the sheath. Other applications may be envisaged, which implement the medical introducer sheath comprising an ultrasonic imaging probe according to the embodiments, in combination with an elongated medical device of the catheter type.

Various embodiments and variants have been described. Those skilled in the art will understand that certain features of these various embodiments and variants could be combined, and other variants will be apparent to those skilled in the art.

Finally, the practical implementation of the described embodiments and variants is within the reach of those skilled in the art based on the functional indications given above.

1. A medical introducer sheath extending in an axial direction between a distal end and a proximal end opposite the distal end, the medical introducer sheath having an internal axial lumen between said distal end and said proximal end and a bendable portion extending to said distal end, the bendable portion comprising:
an ultrasonic imaging probe comprising several ultrasonic transducers distributed in a matrix on a transverse face of the medical introducer sheath at the distal end, around the axial lumen;
an orientation coupler adapted to impart a curvature to the bendable portion;
a sleeve disposed around the orientation coupler, said sleeve comprising a tubular portion and a protruding peripheral portion wound in several protruding turns around the tubular portion, along the axial direction, the tubular portion being comprised between the peripheral portion and the orientation coupler; and
connection strips extending from the ultrasonic transducers in the axial direction toward the proximal end and distributed around the sleeve; and
an elastic structure wound in several turns around the sleeve and the connection strips, the elastic structure running between the protruding turns.

2. The medical introducer sheath according to claim 1, wherein the peripheral portion has a helical shape, the protruding turns corresponding to the turns of the helix, the elastic structure also having a helical shape running between the protruding turns.

3. The medical introducer sheath according to claim 1, wherein the protruding turns of the peripheral portion are protruding rings mutually disjoined and distributed, for example regularly distributed, over several circumferences of the tubular portion along the axial direction, the turns of the elastic structure also being rings mutually disjoined and positioned between the protruding rings.

4. The medical introducer sheath according to claim 1, wherein the pitch between the turns of the elastic structure is substantially equal to the pitch between the protruding turns of the peripheral portion, the turns of the elastic structure being regularly distributed along the axial direction.

5. The medical introducer sheath according to claim 1, wherein the pitch between the turns of the elastic structure is substantially equal to the pitch between the protruding turns of the peripheral portion, the turns of the elastic structure being irregularly distributed along the axial direction, for example with a higher density around a center of the bendable portion.

6. The medical introducer sheath according to claim 1, wherein the axial lumen is sized to receive a catheter in a sliding and/or rotating manner.

7. The medical introducer sheath according to claim 1, wherein the orientation coupler comprises links, for example ball joints, two adjacent links cooperating with each other so as to be able to pivot about at least one axis of rotation.

8. The medical introducer sheath according to claim 7, wherein the orientation coupler comprises linkage cables connected to the links so as to hold the links against each other and to control the pivotal movement of the links, and thus a bending of the bendable portion.

9. The medical introducer sheath according to claim 1, wherein the ultrasonic transducers of the ultrasonic probe are distributed in several concentric crowns around the axial lumen, each crown comprising several transducers distributed in radial sectors along said crown, for example, the number of crowns is greater than 3 and the number of transducers per crown is greater than 30.

10. The medical introducer sheath according to claim 1, wherein the ultrasonic transducers are piezoelectric transducers and are formed by several annular layers extending one over the other around the axial lumen, said annular layers comprising:
a piezoelectric layer metallized on each of its inner and outer faces and divided into several piezoelectric sectors, for example annular and radial sectors;
an impedance matching layer on the piezoelectric layer and divided into several impedance matching sectors, each impedance matching sector being positioned opposite a piezoelectric sector; and
an acoustic attenuation layer beneath the piezoelectric layer.

11. The medical introducer sheath according to claim 10, further comprising an interconnection structure including the connection strips, the interconnection structure further comprising an annular portion disposed between the acoustic attenuation layer and the piezoelectric layer, said annular portion comprising metallic tracks connected to the ultrasonic transducers and extending into the connection strips.

12. The medical introducer sheath according to claim 1, further comprising an annular tip at the distal end, said tip being connected to the orientation coupler, for example to a distal link of said orientation coupler, the ultrasonic transducers being disposed in a circumferential groove of said tip all around the axial lumen.

13. The medical introducer sheath according to claim 1, further comprising an outer envelope around the connection strips, the sleeve and the elastic structure, the outer envelope being, for example, made of a biocompatible material.

14. An intracorporeal exploration and intervention device comprising a medical introducer sheath according to claim 1.

15. The device according to claim 14, further comprising a catheter configured to be introduced into the axial lumen of the medical introducer sheath.

16. The device according to claim 14, further comprising, at the proximal end, a control handle adapted to control a bending of the bendable portion.
